(19) United States
(12) Patent Application Publication
(10) Pub. No.: US 2012/0230965 A1
(43) Pub. Date: Sep. 13, 2012

(54) Novel Microorganism Bacillus megaterium Toha Producing L-Type Poly-Gamma-Glutamic Acid and L-Type Poly-Gamma-Glutamic Acid Produced Thereby

(75) Inventors: Moon-Hee SUNG, Daejeon (KR); Chul Joong KIM, Daejeon (KR); Haryoung POO, Daejeon (KR); Mi-Sun KWAK, Koyang-si (KR); Sung-Bin PARK, Bucheon-si (KR); Dong-Hwan CHOI, Gwangmung-si (KR); Ji Yeon KIM, Daejeon (KR)

(73) Assignees: BIOLEADERS CORPORATION, Daejeon (KR); THE INDUSTRY AND ACADEMIC COOPERATION IN CHUNGNAM NATIONAL UNIVERSITY, Daejeon (KR); KOREA RESEARCH INSTITUTE OF BIOSCIENCE AND BIOTECHNOLOGY, Daejeon (KR); KOOKMIN UNIVERSITY INDUSTRY-ACADEMIC COOPERATION FOUNDATION, Seoul (KR)

(21) Appl. No.: 13/313,972

(22) Filed: Dec. 7, 2011

(30) Foreign Application Priority Data
Dec. 7, 2010 (KR) 10-2010-0124020

(51) Int. Cl. A61K 38/02; A61K 35/74; C07K 14/00; C12N 1/20; C12P 21/02

(52) U.S. Cl. 424/93.46; 435/252.5; 435/71.2; 530/350; 514/1.1

(57) ABSTRACT

The present invention relates to a novel microorganism producing L-type poly-gamma-glutamic acid and an L-type poly-gamma-glutamic acid produced thereby and, more particularly, to Bacillus megaterium Toha (KCTC11752BP) isolated from salt-fermented Toha shrimps (Korean traditional fermented food) and an L-type poly-gamma-glutamic acid produced thereby. The novel microorganism Bacillus megaterium Toha (KCTC11752BP) has the ability to produce L-type poly-gamma-glutamic acid which can be used in high-value-added microbial agents, probiotic agents, immune-activating agents, animal drugs, cosmetics, moisture absorbers, thickeners, biodegradable plastics, etc.

### BACKGROUND OF THE INVENTION

1. Field of the Invention

The present invention relates to a novel microorganism producing L-type poly-gamma-glutamic acid and an L-type poly-gamma-glutamic acid produced thereby and, more particularly, to Bacillus megaterium Toha (KCTC11752BP) isolated from salt-fermented Toha shrimps (Korean traditional fermented food) and an L-type poly-gamma-glutamic acid produced thereby.

2. Background of the Related Art

Poly-gamma-glutamic acid is a viscous polymer consisting of D and L-glutamic acids polymerized through gamma-glutamyl bonds. It is produced by Bacillus sp. strains isolated from Chungkookjang (Korean traditional fermented soybean food prepared using rice-straw), Natto (see Japanese traditional fermented soybean food), Kinema (Nepalese traditional fermented soybean food), etc. Poly-gamma-glutamic acids produced by Bacillus sp. strains are classified according to the stereospecificity of glutamic acid into D-type poly-gamma-glutamic acid consisting mainly of D-glutamic acid, L-type poly-gamma-glutamic acid consisting mainly of L-glutamic acid, and D,L-type poly-gamma-glutamic acid consisting of D- and L-glutamic acid. The D,L-type poly-gamma-glutamic acid is an edible, water-soluble, anionic and biodegradable polymer substance (molecular weight: 100-2,000 kDa), which can be used as a raw material for moisture absorbers, moisturizers, cosmetic raw materials, and as a raw material for preparing biodegradable plastics by synthesis of ester derivatives.

Recently, with respect to the production and utilization of poly-gamma-glutamic acid, studies focused on the material of products substituting for non-biodegradable polymers, the development of heat-resistant plastics by esterification and the production of water-soluble fibers and membranes have been actively conducted, particularly in advanced countries. Also, there have been active studies on changes in the physical properties of poly-gamma-glutamic acid, which result from irradiation with gamma-rays, and on the development and industrialization of hydrogel using crosslinking agents.

For example, the following studies and patents have been reported: the effects of manganese ions on the composition and production of poly-gamma-glutamic acid, the use of poly-gamma-glutamic acid as water-soluble polymers by untrasonic decomposition, and the development of low-water-soluble plastics by the synthesis of ester derivatives (Biosci. Biotechnol. Biochem., 60:1239, 1996); the production of poly-gamma-glutamic acid by Bacillus subtillis, and the use of poly-gamma-glutamic acid as a calcium dissolving agent in healthy foods having an osteoporosis therapeutic effect (see Japanese Patent Laid-Open Publication No. Hei 6-32742); effects of reducing the content of phosphorus in water systems to reduce water contamination (EP 838160); preparation of an absorptive and biodegradable resin having with high gelatination rate and water absorption using poly-gamma-glutamic acid by irradiation of radiation, and the application of the prepared resin to hygienic products (e.g., diapers) and food products and in the horticultural industry (see Japanese Patent Laid-Open Publication Nos. Hei 10-251402, 7-300522 and 6-322358), etc.

In addition, there are reports on the use of poly-gamma-glutamic acid as biodegradable solid fibers or films and molded films by the dissolution, precipitation and drying of poly-gamma-glutamic acid (see Japanese Patent Laid-Open Publication Nos. Hei 7-138364 and Hei 5-117388); and polymers for drug carriers (see Japanese Patent Laid-Open Publication Nos. Hei 6-92870 and Hei 6-256220).

Meanwhile, in Korea, the following researches have been conducted: a basic research on the efficient production of poly-gamma-glutamic acid (see Korean Patent Application Nos. 1997-3404 and 1997-67605); improvement in the physical properties of poly-gamma-glutamic acid; and an application research on the use of poly-gamma-glutamic acid, produced by the Bacillus Natto strain, as a raw material for cosmetic products (conducted by Amore Pacific Co., Ltd., Korea).

It was reported that L-type poly-gamma-glutamic acid forms a specific steric structure, and thus the moisturizing ability thereof increases in the presence of a salt. Thus, the use of L-type poly-gamma-glutamic acid as a new moisturizing material has been reported (ToYoBo CO., LTD. Apr. 6, 2007).

Previously, the present inventors isolated Bacillus subtilis var. chungkookjang (KCTC 0697BP), a salt-tolerant strain, from Chungkookjang, and found that the Bacillus subtilis var. chungkookjang strain produces a poly-gamma-glutamic acid having a content ratio between D- and L-glutamic acids of 40:60 (see Korean Patent Registration No. 0500796).

Bacillus megaterium WH320 is known to produce L-type poly-gamma-glutamic acid, but it is reported that this microbial strain produces L-type poly-gamma-glutamic acid only in high-salt conditions (Shimizu, K. et al, Appl. Environ. Microbiol., 73:2378, 2007; Japanese Patent Laid-Open Publication No. 2007-228957).

Accordingly, the present inventors have made extensive efforts to develop a novel strain producing L-type poly-gamma-glutamic acid and, as a result, have found that Bacillus megaterium Toha, a novel microbial strain isolated from salt-fermented Toha shrimps, produces an L-type poly-gamma-glutamic acid having a high content of L-glutamic acid even in low-salt conditions, thereby completing the present invention.

### SUMMARY OF THE INVENTION

It is an object of the present invention to provide Bacillus megaterium Toha (KCTC 11752BP), a novel microbial strain producing L-type poly-gamma-glutamic acid useable as a new moisturizing material, and an L-type poly-gamma-glutamic acid produced by the strain.

To achieve the above object, the present invention provides a Bacillus megaterium Toha (KCTC 11752BP), which is a novel microorganism.

The present invention also provides a method for preparing poly-gamma-glutamic acid, the method comprising the steps of: culturing Bacillus megaterium Toha (KCTC 11752BP); and isolating poly-gamma-glutamic acid from the cultured Bacillus megaterium Toha (KCTC 11752BP).

The present invention also provides an L-type poly-gamma-glutamic acid, which is produced by Bacillus megaterium Toha (KCTC 11752BP), and has a weight-average molecular weight of about 8900 kDa and a content ratio between D- and L-glutamic acids of about 10:90.

The present invention also provides a probiotic composition containing Bacillus megaterium Toha (KCTC 11752BP) and a poly-gamma-glutamic acid produced by the Bacillus megaterium Toha.

The present invention also provides an immune-enhancing pharmaceutical composition containing an L-type poly-gamma-glutamic acid, wherein the L-type poly-gamma-glutamic acid is produced by Bacillus megaterium Toha (KCTC 11752BP), and has a weight-average molecular weight of about 8900 kDa and a content ratio between D- and L-glutamic acids of about 10:90.

The present invention also provides a functional food containing an L-type poly-gamma-glutamic acid, wherein the L-type poly-gamma-glutamic acid is produced by Bacillus megaterium Toha (KCTC 11752BP), and has a weight-average molecular weight of about 8900 kDa and a content ratio between D- and L-glutamic acids of about 10:90.

The present invention also provides a cosmetic composition containing an L-type poly-gamma-glutamic acid, wherein the L-type poly-gamma-glutamic acid is produced by Bacillus megaterium Toha (KCTC 11752BP), and has a weight-average molecular weight of about 8900 kDa and a content ratio between D- and L-glutamic acids of about 10:90.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a set of transmission electron microscopes of the novel microorganism Bacillus megaterium Toha according to the present invention.

FIG. 2 shows the results of analyzing the homology of the novel microorganism Bacillus megaterium Toha according to the present invention using 16S rDNA.

FIG. 3 is a set of graphs showing the stereospecificities of D- and L-glutamic acids of L-type poly-gamma-glutamic acid in the novel microorganism Bacillus megaterium Toha according to the present invention in the presence of various concentrations of NaCl.

FIG. 4 is a graph showing the macrophage-stimulating effect (induction of TNF-α secretion) of poly-gamma-glutamic acid produced by the novel microorganism Bacillus megaterium Toha.

FIG. 5 is a graph showing a comparison of macrophage-stimulating effect (induction of TNF-α secretion) between a poly-gamma-glutamic acid produced by the novel microorganism Bacillus megaterium Toha and a poly-gamma-glutamic acid produced by the existing Bacillus subtilis var. chungkookjang strain.

FIG. 6 shows the nucleotide sequence of the 16S rDNA gene of the novel microorganism Bacillus megaterium Toha.

### DETAILED DESCRIPTION OF THE PREFERRED EMBODIMENT

Now, a preferred embodiment of the present invention will be descried hereinafter in more detail with reference to the accompanying drawings.

In one aspect, the present invention is directed to a Bacillus megaterium Toha (KCTC 11752BP), which is a novel microorganism.

In the present invention, in order to obtain microorganisms producing L-type poly-gamma-glutamic acid, salt-fermented Toha shrimps which are produced and marketed in Korea were collected and a microbial strain producing L-type poly-gamma-glutamic acid was isolated therefrom. As a result, a Bacillus strain, Bacillus megaterium Toha, was obtained, which produces a poly-gamma-glutamic acid different from that produced by Bacillus subtilis var. chungkookjang isolated from Chungkookjang (Korean traditional fermented soybean food).

The Bacillus megaterium Toha strain according to the present invention produces an edible, water-soluble, anionic and biodegradable L-type poly-gamma-glutamic acid and was isolated and identified in the following manner.

In order to a microbial strain, which produces L-type poly-gamma-glutamic acid, from salt-fermented Toha shrimps among Korean traditional salt-fermented aquatic products, each of salt-fermented Toha shrimp samples was suspended in 0.85% NaCl, and then cultured in a poly-gamma-glutamic acid-producing agar plate medium supplemented with 10% NaCl, and microbial strains producing a viscous substance were isolated from the culture broths. These isolated microbial strains were cultured in poly-gamma-glutamic acid-producing agar liquid media supplemented with 10% NaCl, after which the amount of poly-gamma-glutamic acid produced, the weight-average molecular weight of the produced poly-gamma-glutamic acid, and the ratio between the contents of D- and L-glutamates in the produced poly-gamma-glutamic acid were analyzed, and a microbial strain showing a specific content ratio between D- and L-glutamates was selected.

The selected strain has the following characteristics: it forms a yellow bacterial colony when being cultured in LG agar plate medium; it is a gram-positive bacterium whose cells actively grow in aerobic conditions at 37° C. or higher; and the cells thereof slowly grow at a culture temperature of 55° C. or higher. Also, the strain according to the present invention is a salt-tolerant strain capable of growing even at a NaCl concentration of 10% that is higher than that of general Bacillus subtillis. In addition, the 16S rDNA nucleotide sequence of the isolated strain according to the present invention was analyzed comparatively with the 16S rDNA nucleotide sequences of existing Bacillus sp. strains, and as a result, the isolated strain of the present invention showed a very high 16S rDNA sequence homology of 99.0% with Bacillus megaterium.

The selected strain of the present invention is obtained from salt-fermented Toha shrimps which have been used for edible purposes for a long time and have proven safety. Thus, high-molecular-weight L-type poly-gamma-glutamic acid produced by the strain of the present invention can be safe for use in foods, beverages, health foods, cosmetics, medical drugs, etc.

In another aspect, the present invention is also directed to a method for preparing poly-gamma-glutamic acid, the method comprising the steps of: culturing Bacillus megaterium Toha (KCTC 11752BP); and isolating poly-gamma-glutamic acid from the cultured Bacillus megaterium Toha (KCTC 11752BP).

In still another aspect, the present invention is also directed to an L-type poly-gamma-glutamic acid, which is produced by Bacillus megaterium Toha (KCTC 11752BP), and has a weight-average molecular weight of about 8900 kDa and a content ratio between D- and L-glutamic acids of about 10:90.

In yet another aspect, the present invention also provides a probiotic composition containing Bacillus megaterium Toha (KCTC 11752BP) and a poly-gamma-glutamic acid produced by the Bacillus megaterium Toha.

In a further aspect, the present invention also provides an immune-enhancing pharmaceutical composition containing an L-type poly-gamma-glutamic acid, wherein the L-type poly-gamma-glutamic acid is produced by Bacillus megaterium Toha (KCTC 11752BP), and has a weight-average molecular weight of about 8900 kDa and a content ratio between D- and L-glutamic acids of about 10:90.

The human dose of the immune-enhancing pharmaceutical composition according to the present invention may vary depending on the patient's age, weight and sex, the mode of administration, physical conditions, the route of administration and the severity of the disease being treated. However, for preferred effects, the unit dose of the composition is preferably 0.1-10 g/kg, and this dose is not intended to limit the scope of the present invention in any way. Also, the dose may vary depending on the patient's age, sex, weight and physical conditions.

In a still further aspect, the present invention is also directed to a functional food containing an L-type poly-gamma-glutamic acid, wherein the L-type poly-gamma-glutamic acid is produced by Bacillus megaterium Toha (KCTC 11752BP), and has a weight-average molecular weight of about 8900 kDa and a content ratio between D- and L-glutamic acids of about 10:90.

In a yet further aspect, the present invention is also directed to a cosmetic composition containing an L-type poly-gamma-glutamic acid, wherein the L-type poly-gamma-glutamic acid is produced by Bacillus megaterium Toha (KCTC 11752BP), and has a weight-average molecular weight of about 8900 kDa and a content ratio between D- and L-glutamic acids of about 10:90.

The cosmetic composition according to the present invention may contain antioxidants, binders, bulking agents, chelating agents, colorants, emollients, emulsion stabilizers, film formers, fillers, fragrance components, gelling agents, hair conditioning agents, hair fixatives, humectants, plasticizers, preservatives, skin conditioning agents, solvents, sunscreen agents, surfactants, ultraviolet light absorbers, viscosity controlling agents, and waxes.

The novel isolated strain “Bacillus megaterium Toha” according to the present invention produced high-molecular-weight L-type poly-gamma-glutamic acid even at low salt concentrations, unlike existing Bacillus megaterium strains that produce at high salt concentrations. Based on the above-described results, the strain of the present invention was named “Bacillus megaterium Toha” and deposited in the Korean Collection for Type Cultures (KCTC), the Korea Research Institute of Bioscience and Biotechnology (KRIBB) (52 Eoeun-dong, Yuseong-gu, Daejeon 305-333, Korea), under the accession number KCTC 11752BP.

The molecular weight of poly-gamma-glutamic acid produced by the Bacillus megaterium Toha strain of the present invention was as high as about 8,900 kDa, which was similar to that of poly-gamma-glutamic acid produced by existing B. subtilis Chungkookjang known as a strain producing high-molecular-weight poly-gamma-glutamic acid. Also, the poly-gamma-glutamic acid produced by the Bacillus megaterium Toha strain of the present invention had a uniform molecular weight distribution.

Furthermore, the Bacillus megaterium Toha strain of the present invention was cultured on LG solid media supplemented with various concentrations of NaCl, and the ratio between the contents of D- and L-glutamic acids in the poly-gamma-glutamic acid was examined. As a result, it was found that the Bacillus megaterium Toha strain of the present invention produced high-molecular-weight L-type poly-gamma-glutamic acid even at low salt concentrations, unlike a Bacillus megaterium strain that produces L-type poly-gamma-glutamic acid at high salt concentrations (Shimizu, K. et al, Appl. Environ. Microbiol., 73:2378, 2007; Japanese Patent Laid-Open Publication No. 2007-228957).

In addition, it was found that L-type poly-gamma-glutamic acid produced by the Bacillus megaterium Toha strain of the present invention induced the macrophage secretion of TNF-a as an index of the activation of macrophages playing an important role in immune responses.

### EXAMPLES

Hereinafter, the present invention will be described in further detail with reference to examples. It will be obvious to those skilled in the art that these examples are illustrative purposes only and are not to be construed to limit the scope of the present invention.

### Example 1

### Isolation of Microorganisms Producing L-Type Poly-Gamma-Glutamic Acid

In order to isolate a strain, which produces L-type poly-gamma-glutamic acid, from salt-fermented Toha shrimps among Korean traditional fermented foods, each of salt-fermented Toha shrimp samples was suspended in 0.85% NaCl, and then plated on a poly-gamma-glutamic acid-producing agar plate medium (L-glutamic acid 2%, glucose 5%, MgSO₄ 7H₂O 0.5%, (NH₄)₂SO₄ 1%, KH₂PO₄ 0.27%, Na₂HPO₄ 0.4%, NaCl 10%, thiamin 0.0002%, and agar 2%) supplemented with 10% NaCl. Then, each sample was cultured in an incubator at 37° C., and microbial colonies producing a viscous substance were isolated. Each of these isolated strains was inoculated onto a poly-gamma-glutamic acid-producing solid medium supplemented with 10% NaCl and was cultured at 37° C. Then, the weight-average molecular weight of the produced poly-gamma-glutamic acid (PGA) and the ratio between the contents of D- and L-glutamic acids in PGA were examined, and a strain having a higher content of L-glutamic acid was finally selected.

### Example 2

### Identification of Strain

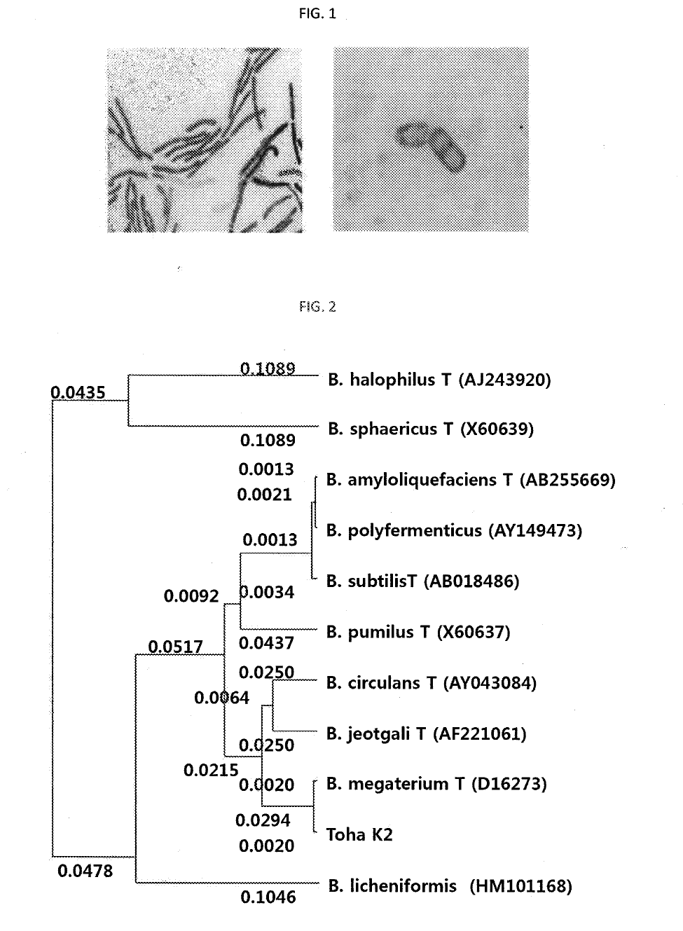

The selected strain was identified by analyzing the biochemical characteristics of the strain using an AlP system, observing the morphological characteristics of the strain with a transmission electron microscope, and analyzing the 16S rDNA of the strain. It could be seen that the selected strain is a gram-positive, long-rod-shaped bacillus and is an aerobic spore-forming Bacillus sp. strain (see FIG. 1).

For accurate identification of the selected strain, the sequencing of the 16S rDNA gene was performed according to a conventional method. For this purpose, DNA was isolated from the selected strain, and the 16S rDNA gene was amplified by PCR using an N-terminal primer (5′-AGAGTTTGATCCTGGCTCAG-3′: SEQ ID NO: 2) and a C-terminal primer (5′-AGAAAGGAGGTGATCCAGCC-3′: SEQ ID NO: 3). The PCR amplification product was cloned into a plasmid pGEM-T vector, and the entire nucleotide sequence of the 16S rDNA gene was determined.

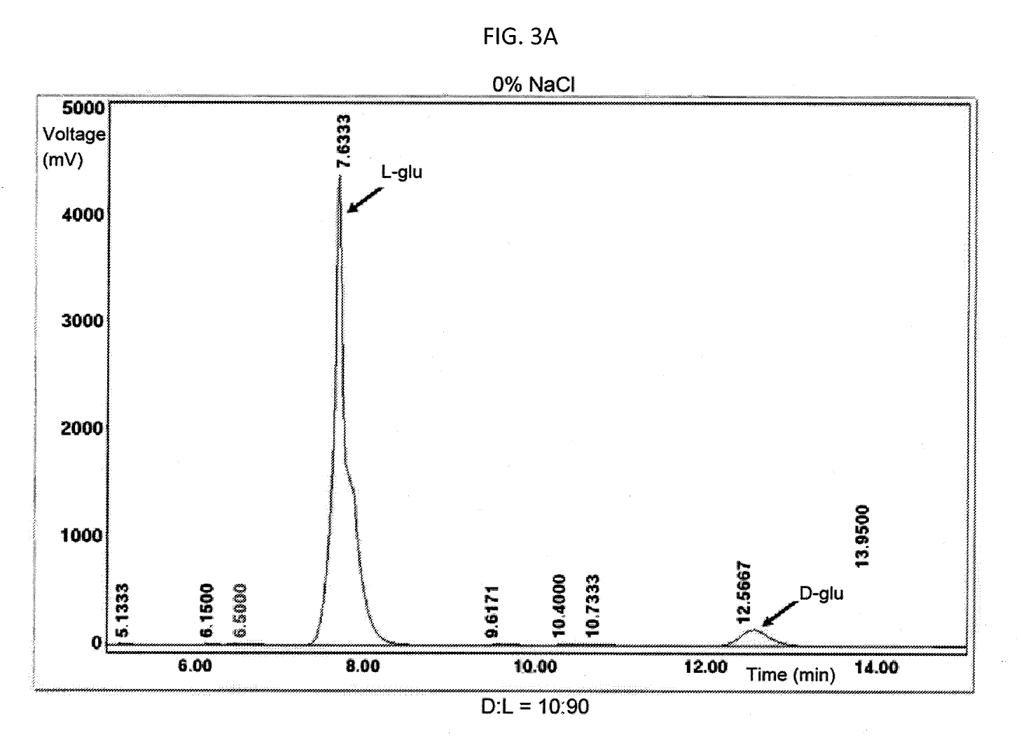

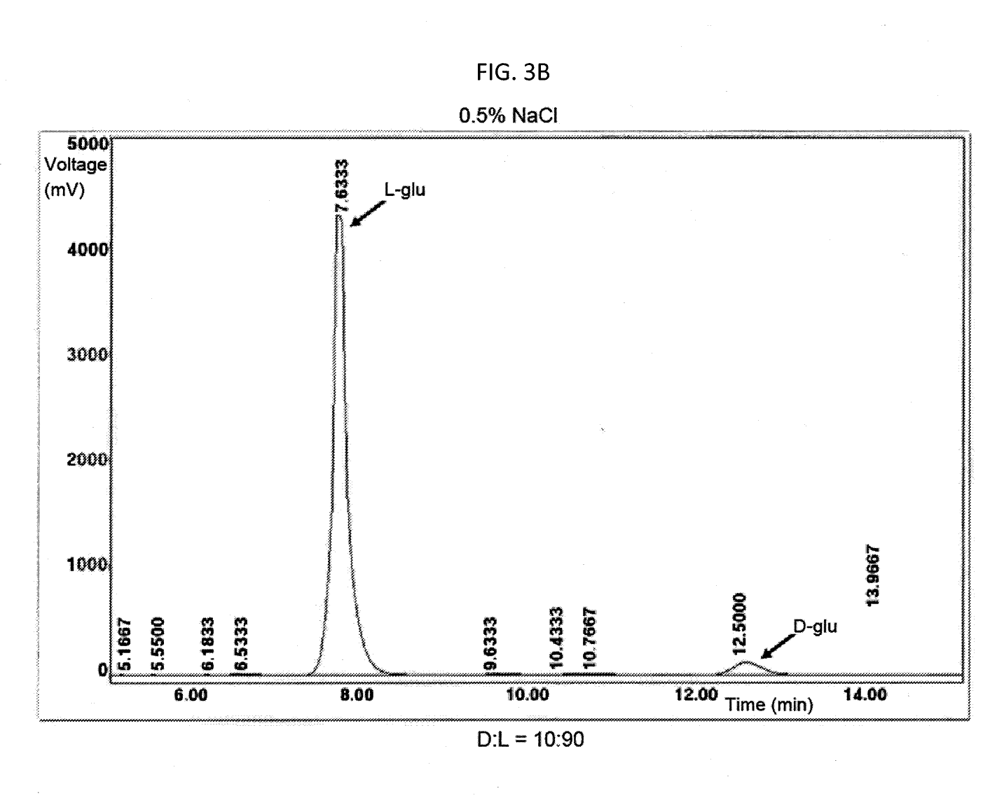

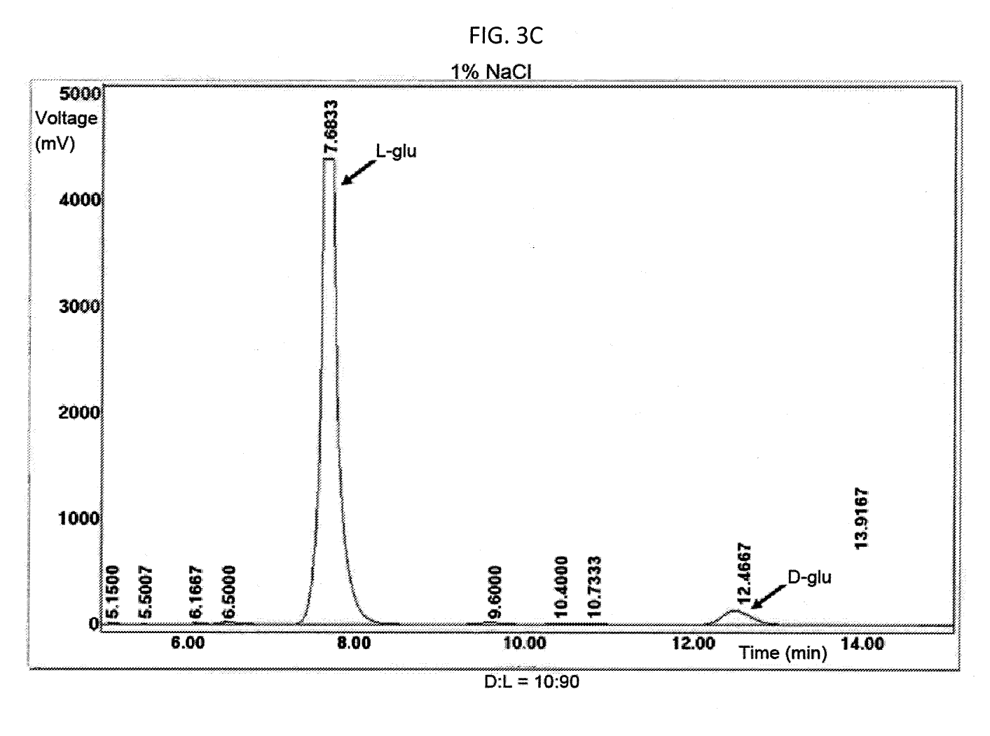

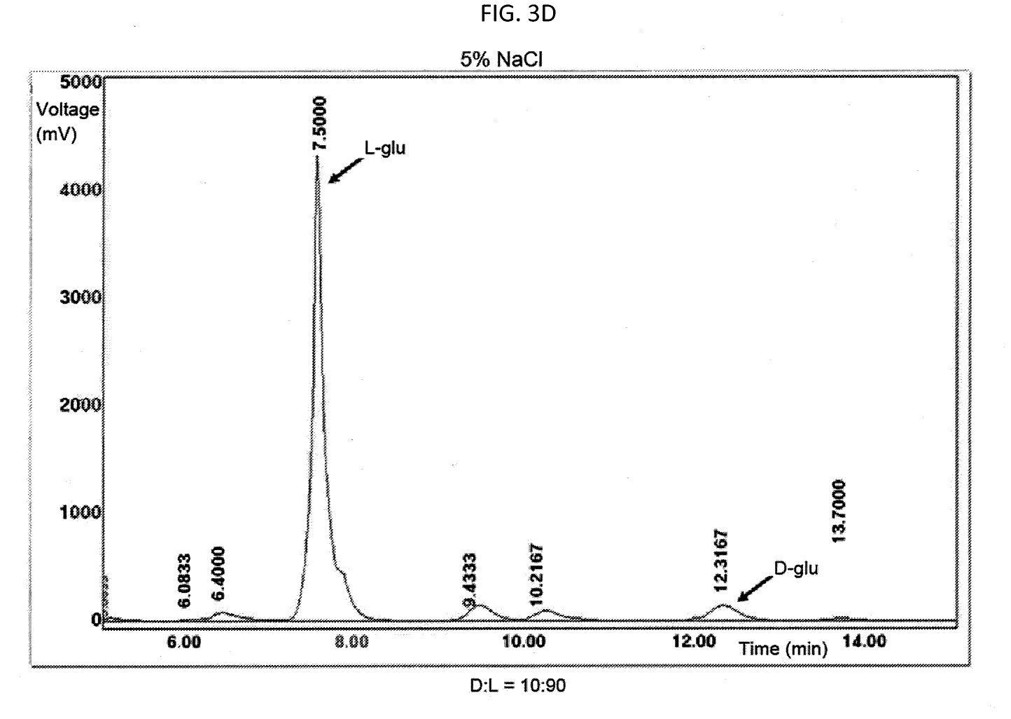

The comparison of homology between the 16S rDNA nucleotide sequence (SEQ ID NO: 1) of the selected microbial strain and those of various microbial strains known in the prior art was performed. As a result, as can be seen in FIG. 3, the selected strain showed a sequence homology of 99.0% or higher with Bacillus megaterium. Based on the above-described results, the strain of the present invention was named “Bacillus megaterium Toha” and deposited in the Korean Collection for Type Cultures (KCTC), the Korea Research Institute of Bioscience and Biotechnology (KRIBB) (52 Eoeun-dong, Yuseong-gu, Daejeon 305-333, Korea), under the accession number KCTC 11752BP).

### Example 3

### Comparison of Molecular Weight and Viscosity Between Poly-Gamma-Glutamic Acids

The Bacillus megaterium Toha strain was cultured on LG solid medium (L-glutamic acid 2%, glucose 5%, MgSO₄ 7H₂O 0.5%, (NH₄)₂SO₄ 1%, KH₂PO₄ 0.27%, Na₂HPO₄ 0.4%, NaCl 0.5%-10%, thiamin 0.0002%, and agar 2%) for 5 days, after which poly-gamma-glutamic acid was isolated from the strain according to the above-described method, and the molecular weight thereof was analyzed using gel permeation chromatography (Viscotek GMPWXL (7.6 mm ID×300 mm)). The gel permeation chromatography was performed using 0.1M NaNO₃ solution as a solvent at a solvent flow rate of 0.8 ml/min and a column oven temperature of 40° C. Also, polyacrylamide (American Polymer Standards Corporation, USA) was used as a standard material, and a refractive index meter was used to measure the molecular weight of the poly-gamma-glutamic acid.

As a result, as can be seen in Table 1 below, the poly-gamma-glutamic acid produced by the Bacillus megaterium Toha strain had an Mw (weight-average molecular weight) of about 8,900 kDa and a polydispersity of about 4.2. Thus, it could be seen that the poly-gamma-glutamic acid produced by the Bacillus megaterium Toha strain had a high molecular weight which is similar to that of the poly-gamma-glutamic acid produced by B. subtilis chungkookjang reported to be a strain producing high-molecular-weight poly-gamma-glutamic acid, and the molecular weight distribution thereof was also uniform.

TABLE 1
Molecular weights of poly-gamma-glutamic acids

| | Weight-average | |
|---|---|---|
| PGA-producing | molecular weight | |
| strains | (kDa) | polydipersity |
| B. subtilis | 7,000 | 5.3 |
| Chungkookjang | | |
| B. megaterium Toha | 8,900 | 4.2 |
| B. megaterium WH320 | 1,000-2,000 | |

Also, the 8,900-kDa poly-gamma-glutamic acid produced by the Bacillus megaterium Toha strain was heated to prepare samples having various molecular weights, and the viscosities of the prepared samples were compared with the viscosity of the poly-gamma-glutamic acid produced by B. subtilis chungkookjang. As a result, it could be seen that the two poly-gamma-glutamic acids having the same molecular weight had similar viscosities.

TABLE 2
Viscosities of poly-gamma-glutamic acids (1% in DW)

| | Viscosity of PGA | |
|---|---|---|
| | produced by | Viscosity of PGA |
| Weight-average | B. subtilis | produced by |
| molecular weight | Chungkookjang | B. megaterium Toha |
| 8,000 kDa | 670 | 680 |
| 5,000 kDa | 500 | 490 |
| 1,500 kDa | 90 | 80 |
| 500 kDa | 27 | 25 |
| 50 kDa | 6 | 5 |

### Example 4

### Examination of Stereospecificity of D- and L-Glutamic Acids in Poly-Gamma-Glutamic Acid

The composition of D- and L-glutamic acids in the poly-gamma-glutamic acid produced by the isolated strain of the present invention was examined.

In order to examine the ratio between the contents of D- and L-glutamic acids in the poly-gamma-glutamic acid produced by the isolated strain of the present invention, the isolated strain of the present invention was inoculated into a poly-gamma-glutamic acid-producing medium and cultured in an incubator at 37° C. for 48 hours, after which poly-gamma-glutamic acid was isolated from the strain in the same manner as described above. The resulting poly-gamma-glutamic acid was degassed by adding 0.1N hydrochloric acid thereto, and then hydrolyzed at 105° C. for 10 hours.

The composition of amino acids in the hydrolysis product was analyzed by a Nova-pak C₁₈ (Waters) HPLC column using a 50 mM triethyl amine-H₃PO₄ (pH 3.7) solvent containing 18% acetonitrile. Separation of the stereoisomers was performed by derivatizing the amino terminal regions of the D- and L-glutamic acids using FDAA, and then the D- and L-glutamic acids in the poly-gamma-glutamic acid were quantified using a UV detector at 340 nm according to standard curves of D- and L-glutamic acids. The contents of D- and L-glutamic acids in the produced poly-gamma-glutamic acid were examined as described above. As a result, as can be seen in Table 3 below, the ratio between the contents of D- and L-glutamic acids in the poly-gamma-glutamic acid produced by the isolated strain of the present invention was about 10:90, suggesting that the strain of the present invention produced L-type poly-gamma-glutamic acid.

TABLE 3
Ratio between contents of D- and L-glutamates in poly-
gamma-glutamic acids

Content ratio of D- and L-
glutamates

| PGA-producing strains | D-glutamate | L-glutamate |
|---|---|---|
| B. subtilis | 60 | 40 |
| Chungkookjang | | |
| B. megaterium Toha | 10 | 90 |

### Example 5

### Examination of Stereospecificities in D- and L-Glutamic Acids in Poly-Gamma-Glutamic Acid at Various NaCl Concentrations

In order to examine whether the ratio between the contents of D- and L-glutamic acids in the poly-gamma-glutamic acid produced by the Bacillus megaterium Toha strain changes with an increase in NaCl concentration, the isolated strain of the present invention was cultured on LG solid media supplemented with various concentrations of NaCl in an incubator at 37° C. for 48 hours. Then, the ratio between the contents of D- and L-glutamic acids in the poly-gamma-glutamic acid isolated from the cultured strain was examined in the same manner as described above. As a result, as can be seen in FIG. 3, the isolated strain of the present invention produced high-molecular-weight L-type poly-gamma-glutamic acid even at low salt concentrations, unlike a Bacillus megaterium strain that produces L-type poly-gamma-glutamic acid at high salt concentrations (Shimizu, K. et al, Appl. Environ. Microbiol., 73:2378, 2007; Japanese Patent Laid-Open Publication No. 2007-228957).

### Example 6

### Induction of TNF-α Secretion in Macrophages by Poly-Gamma-Glutamic Acid

In order to examine the effect of the L-type poly-gamma-glutamic acid produced by the Bacillus megaterium Toha strain of the present invention on the activation of macrophages playing an important role in the primary immune response of the immune system, the following experiment was carried out.

As an index of the activation of macrophages, the secretion of TNF-α (major cytokine mediating innate immunity) from macrophages was examined.

For this purpose, the Balb/c mouse macrophage cell line RAW 264.7 (ATCC TIB-71) was suspended in DMEM medium (100 U/ml penicillin-streptomycin; containing 10% FBS), after which the cells were dispensed into a 6-well plate at a cell density of 5×10⁵ cell/well and cultured in a CO₂ incubator for 12 hours. Then, poly-gamma-glutamic acid diluted in FBS-free DMEM medium at a concentration of 0.1% was added to the cells which were then cultured for 24 hours. Then, the culture supernatant of each well was collected, and the amount of TNF-α in the collected culture supernatant was measured using an ELISA kit (BD Bioscience, USA). A 96-well plate was coated with an anti-mouse monoclonal antibody against TNF-α at 4° C. for 12 hours, and then 100 μl of a standard solution and 100 μl of the culture supernatant were added thereto and allowed to react at room temperature for about 1 hour. Then, each well was washed three time with washing buffer (250 μl/well), after which 100 μl of a biotinylated anti-mouse polyclonal antibody (primary antibody) against TNF-α was added thereto and allowed to react at room temperature for 1 hours, and then each well was washed three times with washing buffer (250 μl/well). Then, 100 μl of an avidin-horseradish peroxidase conjugate as secondary antibody was added to each well and allowed to react at room temperature for 1 hour. Then, each well was washed three times and allowed to react with a TMB color-developing agent for 15 minutes, and then 50 μl of a stop solution was added thereto to stop the color development. Then, the absorbance at 450 nm was measured with an ELISA reader, thereby analyzing the amount of TNF-α. As a positive control, lipopolysaccaride (LPS, E. coli) was added at a concentration of 1 ng/ml.

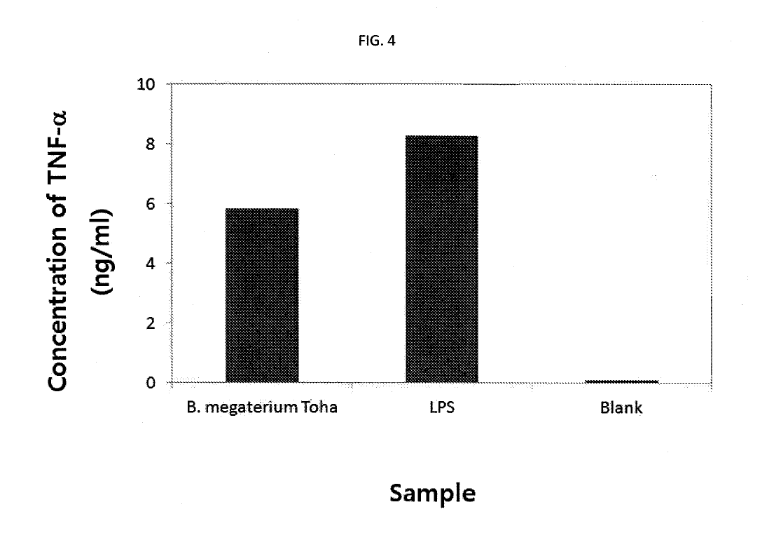

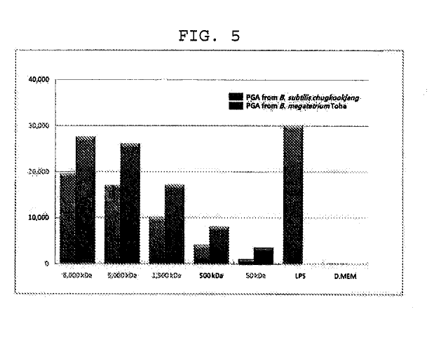

As a result, it could be seen that the poly-gamma-glutamic acid had the effect of stimulating macrophages (see FIG. 4), suggesting that the L-type poly-gamma-glutamic acid produced by the Bacillus megaterium Toha strain of the present invention induces the macrophage secretion of TNF-α as an index for the activation of macrophages playing an important role in immune responses.

In addition, the ability to stimulate macrophages to secrete TNF-α was compared between the L-type poly-gamma-glutamic acid produced by the Bacillus megaterium Toha strain of the present invention and the poly-gamma-glutamic acid produced by B. subtilis chungkookjang. As a result, as can be seen in FIG. 5, the ability to stimulate macrophages to secrete TNF-α was 1.4 times higher in the L-type poly-gamma-glutamic acid produced by the Bacillus megaterium Toha strain than in the poly-gamma-glutamic acid produced by existing B. subtilis chungkookjang, at a molecular weight of 8,000 kDa, and 1.5 times higher at a molecular weight of 5,000 kDa, and also 1.7 times higher at a molecular weight of 1,500 kDa.

As described above, the novel microorganism Bacillus megaterium Toha (KCTC11752BP) according to the present invention has the ability to produce L-type poly-gamma-glutamic acid which can be used in high-value-added cosmetics, moisture absorbers, biodegradable plastics, etc.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

[Depository Institution]

Name of Depository Institution: Korean Collection for Type Cultures

Accession Number: KCTC11752BP

Date of Deposition: Aug. 25, 2010

What is claimed is:

1. A Bacillus megaterium Toha (KCTC 11752BP), which is a novel microorganism.

2. The Bacillus megaterium Toha (KCTC 11752BP) of claim 1, which is derived from fermented foods.

3. The Bacillus megaterium Toha (KCTC 11752BP) of claim 1, which comprises a 16S rDNA sequence represented by SEQ ID NO: 1.

4. The Bacillus megaterium Toha (KCTC 11752BP) of claim 1, which produces an L-type poly-gamma-glutamic acid in a low-salt state.

5. A method for preparing poly-gamma-glutamic acid, the method comprising the steps of: culturing the Bacillus megaterium Toha (KCTC 11752BP) of claim 1; and isolating poly-gamma-glutamic acid from the cultured Bacillus megaterium Toha (KCTC 11752BP).

6. An L-type poly-gamma-glutamic acid, which is produced by Bacillus megaterium Toha (KCTC 11752BP), and has a weight-average molecular weight of about 8900 kDa and a content ratio between D- and L-glutamic acids of about 10:90.

7. A probiotic composition containing Bacillus megaterium Toha (KCTC 11752BP) and a poly-gamma-glutamic acid produced by the Bacillus megaterium Toha.

8. An immune-enhancing pharmaceutical composition containing an L-type poly-gamma-glutamic acid, wherein the L-type poly-gamma-glutamic acid is produced by Bacillus megaterium Toha (KCTC 11752BP), and has a weight-average molecular weight of about 8900 kDa and a content ratio between D- and L-glutamic acids of about 10:90.

9. A functional food containing an L-type poly-gamma-glutamic acid, wherein the L-type poly-gamma-glutamic acid is produced by Bacillus megaterium Toha (KCTC 11752BP), and has a weight-average molecular weight of about 8900 kDa and a content ratio between D- and L-glutamic acids of about 10:90.

10. A cosmetic composition containing an L-type poly-gamma-glutamic acid, wherein the L-type poly-gamma-glutamic acid is produced by Bacillus megaterium Toha (KCTC 11752BP), and has a weight-average molecular weight of about 8900 kDa and a content ratio between D- and L-glutamic acids of about 10:90.


SEQUENCE LISTING (3 sequences)

SEQ ID NO: 1 - DNA, 1470 bp, Bacillus megaterium Toha
aacgctggcggcgtgcctaatacatgcaagtcgagcgaactgattagaagcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggcccaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacaagagtaactgcttgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaaggtgggacagatgattggggtg

SEQ ID NO: 2 - DNA, 20 bp, Artificial
  primer
agagtttgatcctggctcag

SEQ ID NO: 3 - DNA, 20 bp, Artificial
  primer
agaaaggaggtgatccagcc